(19) United States
(12) Patent Application Publication
(10) Pub. No.: US 2023/0104885 A1
(43) Pub. Date: Apr. 6, 2023

(54) YEAST AS A RAW MATERIAL FOR ANIMAL PRODUCT SUBSTITUTES

(71) Applicant: YEAP LTD., Ashdod (IL)

(72) Inventors: Didier TOUBIA, Raanana (IL); Dominik GRABINSKI, San Francisco, CA (US)

(73) Assignee: YEAP LTD., Ashdod (IL)

(21) Appl. No.: 17/801,412

(22) PCT Filed: Feb. 22, 2021
(86) PCT No.: PCT/IL2021/050205

§ 371 Date: Aug. 22, 2022

Related U.S. Application Data
(60) Provisional application No. 62/980,186, filed on Feb. 22, 2020.

(51) Int. Cl. A23J 3/20; C12N 1/16; A23J 3/22; A23J 3/26; A23J 3/14; A23L 31/10

(52) U.S. Cl. CPC A23J 3/20; A23J 3/14; A23J 3/26; A23J 3/227; A23L 31/10; C12N 1/16; A23V 2002/00

(57) ABSTRACT

Disclosed herein is an extrudate comprising a yeast material wherein the yeast material constitutes between 35% and 100% by dry weight of the extrudate. Further, compositions and food products comprising same are disclosed. Further methods for improving the flavor of a yeast material, by adjusting its pH are also disclosed.

### CROSS REFERENCE TO RELATED APPLICATIONS

This application claims the benefit of priority to U.S. Provisional Pat. Application No. 62/980,186 filed Feb. 22, 2020 entitled “YEAST AS A RAW MATERIAL FOR ANIMAL PRODUCT SUBSTITUTES”, the contents of which are incorporated herein by reference in their entirety.

### FIELD OF THE INVENTION

The present invention is directed to yeast extrudates and methods of manufacturing, and using the extrudates in the preparation of food products.

### BACKGROUND OF THE INVENTION

As the human population continues to increase, there’s a growing need for additional food sources alternatively to meat and dairy sources, particularly food sources that are inexpensive to produce but provide a good nutritional value. Moreover, meat production, contributes significantly to the release of greenhouse gases, and there’s a need for new foodstuffs that are equally tasty and nutritious yet less harmful to the environment to produce.

Consumers are increasingly looking for quality, natural ingredients and authenticity. However, meat substitutes are seen as highly processed products. There is still a great need for food product alternatives that can provide the taste, texture, and sensorial experience of traditional animal products, while using an affordable, widely available sources that are naturally rich in proteins.

### SUMMARY OF THE INVENTION

In one aspect of the invention, there is provided an extrudate comprising a yeast material, wherein the yeast material constitutes between 35% and 100% by dry weight of the extrudate.

In some embodiments, the yeast material comprises a protein content between 10% and 90% by weight of the yeast material.

In some embodiments, the extrudate comprises at least 50% by weight of the yeast material.

In some embodiments, the extrudate comprises between 50% and 99% by weight of the yeast material.

In some embodiments, the extrudate comprises between 1% and 15% weight per weight (w/w) of water.

In some embodiments, the extrudate further comprises between 10% and 40% (w/w) of a starch, a cereal, a grain, a legume, or any combination thereof.

In some embodiments, the extrudate further comprises between 30% and 75% (w/w) of an additional protein, wherein the additional protein is a plant protein selected from the group consisting of: vegetable protein, a legume protein, a seed protein, a grain protein, a tuber protein, a root protein, a fruit protein, hemp protein, a nut protein, an algae protein, a seaweed protein, and any combination thereof.

In some embodiments, the extrudate is characterized by a fibrous texture.

In some embodiments, the extrudate comprises between 30% and 75% (w/w) of water.

In some embodiments, the extrudate comprises between 0.01% and 1.5% (w/w) of an expansion regulating agent.

In some embodiments, the expansion regulating agent comprises calcium carbonate.

In some embodiments, the extrudate further comprises a thickening agent, an emulsifier, coloring agent, flavoring agent, flavor masking agent, or any combination thereof.

In some embodiments, the yeast material is derived from downstream food-related industries.

In some embodiments, the yeast material is selected from the group consisting of: a whole yeast, a yeast biomass, a yeast filtrate, a yeast concentrate, any fraction thereof, and any combination thereof.

In another aspect of the invention, there is provided a food product comprising the extrudate of the present invention.

In some embodiments, the food product consists essentially of the extrudate of the present invention.

In another aspect of the invention, there is provided a food product comprising between 35% and 100% by dry weight of a yeast material, wherein the yeast material is selected from the group consisting of: a whole yeast, a yeast biomass, a yeast filtrate, a yeast concentrate, a yeast extrudate, any fraction thereof, and any combination thereof.

In some embodiments, the food product is characterized as being suitable for use as an equivalent product to meat, eggs, dairy products, or any combination thereof.

In some embodiments, the product is in the form of a nugget, a breast, a steak, or a minced meat-like product.

In another aspect of the invention, there is provided a method for improving the flavor of a yeast material, comprising contacting the yeast material with an agent, wherein the agent is characterized by being able to adjust the pH of the yeast material to a pH ranging from 4 to 7.5.

In some embodiments, the improving comprises reducing yeast taste, off-flavor of yeast taste, enhance meat flavor, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-B include pictures of the high moisture extrudate 3 (HME3);

FIGS. 2A-H include pictures of the low moisture extrudates LME1 (FIG. 2A), LME2 (FIG. 2B), LME3 (FIG. 2C), LME4 (FIG. 2D), LME5 (FIG. 2E), LME6 (FIG. 2F), LME7 (FIG. 2G) and LME9 (FIG. 2H);

FIGS. 3A-F include graphs showing sensory properties: stickiness (FIG. 3A), chicken similarity (FIG. 3B), intensity of taste (FIG. 3C), lingering of taste (FIG. 3D), umami taste (FIG. 3E) and off-flavor (FIG. 3F) of yeast extrudate compared to soy, pea, wheat protein extrudate and real chicken breast; and

FIGS. 4A-G include graphs showing sensory properties: chewiness (FIG. 4A), stickiness (FIG. 4B), chicken flavor (FIG. 4C), intensity of taste (FIG. 4D), lingering of taste (FIG. 4E), umami taste (FIG. 4F) and off-flavor (FIG. 4G) of patty comprising different low moisture yeast extrudates compared to soy, and real chicken patty.

### DETAILED DESCRIPTION OF THE INVENTION

The present invention is directed to an extrudate comprising a yeast material, wherein the yeast material constitutes between 35% and 100% by dry weight of the extrudate.

The present invention is also directed to a food product consisting essentially of a yeast material.

The present invention is based, in part, on the finding that food products consisting essentially of a yeast material, or comprising an extrudate as described herein are characterized by a taste and/or texture identical to a corresponding meat, egg, or dairy derived product. In some embodiments, food products consisting essentially of a yeast material, or comprising an extrudate as described herein are characterized by a taste and/or texture indistinguishable from the a taste and/or texture of a corresponding meat, egg or dairy derived product.

The present invention is also directed to a method for improving the flavor of a yeast material, by adjusting its pH to a pH ranging from 4 to 7.5.

### Extrudate

According to some embodiments, the present invention provides an extrudate comprising a yeast material wherein the yeast material constitutes between 35% and 100%, 35% and 99%, 30% and 99%, 45% and 99%, 35% and 98%, 45% and 98%, 35% and 90%, 45% and 90%, 35% and 85%, 45% and 85%, 35% and 70%, 45% and 70%, 35% and 65%, 45% and 65%, 45% and 60%, or between 35% and 50% by dry weight of the extrudate, including any range therebetween. Each possibility represents a separate embodiment of the invention.

In some embodiments, the yeast material comprises a protein content between 10% and 90%, 12% and 90%, 15% and 90%, 20% and 90%, 50% and 90%, 50% and 90%, 10% and 85%, 12% and 85%, 15% and 85%, 20% and 85%, 50% and 85%, 50% and 85%, 10% and 80%, 12% and 80%, 15% and 80%, 20% and 80%, 50% and 80%, 50% and 80%, 10% and 75%, 12% and 75%, 15% and 75%, 20% and 75%, 50% and 75%, or between 50% and 75%, by weight of the yeast material, including any range therebetween. Each possibility represents a separate embodiment of the invention.

In some embodiments, the yeast material comprises a nitrogen content between 1% and 15%, 4% and 15%, 5% and 15%, 6% and 15%, 7% and 15%, 1% and 10%, 4% and 10%, 5% and 10%, 6% and 10%, 7% and 10%, 4% and 7%, or between 5% and 7%, by weight of the yeast material, including any range therebetween. Each possibility represents a separate embodiment of the invention.

In some embodiments, the extrudate comprises at least 50%, at least 55%, at least 60%, at least 65%, at least 70%, at least 75%, by weight of the yeast material, including any range therebetween. Each possibility represents a separate embodiment of the invention.

In some embodiments, the extrudate comprises between 50% and 99%, between 55% and 99%, between 60% and 99%, between 65% and 99%, 50% and 95%, between 55% and 95%, between 60% and 95%, between 65% and 95%, 50% and 90%, between 55% and 90%, between 60% and 90%, between 65% and 90%, between 50% and 70%, between 55% and 70%, between 60% and 70%, between 65% and 70%, 50% and 69%, between 55% and 69%, between 60% and 69%, between 65% and 69%, 50% and 65%, or between 55% and 65%, by weight of the yeast material, including any range therebetween. Each possibility represents a separate embodiment of the invention.

In some embodiments, the extrudate comprises between 1% and 15%, between 5% and 15%, 7% and 15%, 9% and 15%, 10% and 15%, 1% and 12%, 5% and 12%, 7% and 12%, 9% and 12%, or between 10% and 12% weight per weight (w/w) of water, including any range therebetween. Each possibility represents a separate embodiment of the invention.

According to some embodiments, the present invention provides a low moisture extrudate comprising between 50% and 99%, by weight of a yeast material, and between 1% and 15% (w/w) of water. In some embodiments, a low moisture extrudate comprising 50% and 99%, by weight of a yeast material, comprises a protein content between 45% and 90%, 45% and 80%, 45% and 75%, 50% and 75%, 55% and 75%, 60% and 75%, 45% and 70%, 50% and 70%, 55% and 70%, 60% and 70%, 45% and 65%, 50% and 65%, 55% and 65%, by weight of the yeast material, including any range therebetween. In some embodiments, the present invention provides an extrudate comprising between 50% and 99%, by weight of a yeast material, and between 1% and 15% (w/w) of water. In some embodiments, an extrudate comprising 50% and 99%, by weight of a yeast material, comprises a protein content between 45% and 90%, 45% and 80%, 45% and 75%, 50% and 75%, 55% and 75%, 60% and 75%, 45% and 70%, 50% and 70%, 55% and 70%, 60% and 70%, 45% and 65%, 50% and 65%, 55% and 65%, by weight of the yeast material, including any range therebetween. Each possibility represents a separate embodiment of the invention. In some embodiments, the protein content is calculated by conversion of the nitrogen content. In some embodiments, the protein content is calculated by using 6.25 nitrogen-protein conversion coefficient.

In some embodiments, the extrudate further comprises between 10% and 40% (w/w), 12% and 40% (w/w), 15% and 40% (w/w), 20% and 40% (w/w), 25% and 40% (w/w), 10% and 35% (w/w), 12% and 35% (w/w), 15% and 35% (w/w), 20% and 35% (w/w), or between 25% and 35% (w/w), of a starch, a cereal, a grain, a legume, or any combination thereof.

In some embodiments, the low moisture extrudate further comprises between 10% and 40% (w/w), 12% and 40% (w/w), 15% and 40% (w/w), 20% and 40% (w/w), 25% and 40% (w/w), 10% and 35% (w/w), 12% and 35% (w/w), 15% and 35% (w/w), 20% and 35% (w/w), or between 25% and 35% (w/w), of a starch, flour, or both. Non-limiting examples of suitable starches include maltodextrin, inulin, fructooligosaccharides, pectin, carboxymethyl cellulose, guar gum, corn starch, oat starch, potato starch, rice starch, pea starch, and wheat starch. Non-limiting examples of suitable flours include amaranth flour, oat flour, quinoa flour, rice flour, rye flour, sorghum flour, soy flour, wheat flour, and corn flour.

According to some embodiments, the present invention provides an extrudate comprising a yeast material wherein the yeast material constitutes between 35% and 100%, 35% and 99%, 30% and 99%, 45% and 99%, 35% and 98%, 45% and 98%, 35% and 90%, 45% and 90%, 35% and 85%, 45% and 85%, 35% and 70%, 45% and 70%, 35% and 65%, 45% and 65%, 45% and 60%, or between 35% and 50% by dry weight of the extrudate, including any range therebetween. Each possibility represents a separate embodiment of the invention.

In some embodiments, the extrudate further comprises between 30% and 75% (w/w), 35% and 75% (w/w), 39% and 75% (w/w), 40% and 75% (w/w), 45% and 75% (w/w), 50% and 75% (w/w), 55% and 75% (w/w), 30% and 70% (w/w), 35% and 70% (w/w), 39% and 70% (w/w), 40% and 70% (w/w), 45% and 70% (w/w), 50% and 70% (w/w), 55% and 70% (w/w), 30% and 60% (w/w), 35% and 60% (w/w), 39% and 60% (w/w), 40% and 60% (w/w), 45% and 60% (w/w), 50% and 60% (w/w), 30% and 55% (w/w), 35% and 55% (w/w), 39% and 55% (w/w), 40% and 55% (w/w), or between 45% and 55% (w/w) of an additional protein, including any range therebetween. Each possibility represents a separate embodiment of the invention.

In some embodiments, the additional protein is a plant protein selected from the group consisting of: vegetable protein, a legume protein, a seed protein, a grain protein, a tuber protein, a root protein, a fruit protein, hemp protein, a nut protein, an algae protein, a seaweed protein, and any combination thereof. In some embodiments, the extrudate is devoid of soy.

In some embodiments, the extrudate further comprises between 1% and 25% (w/w), 1% and 15% (w/w), 1% and 10% (w/w), 1% and 5% (w/w), 5% and 25% (w/w), 5% and 15% (w/w), or between 5% and 10% (w/w) of a fiber. Non-limiting examples of a fiber according to the present invention include bamboo fiber, barley bran, carrot fiber, citrus fiber, corn bran, soluble dietary fiber, insoluble dietary fiber, oat bran, pea fiber, rice bran, head husks, soy fiber, soy polysaccharide, wheat bran, and wood pulp cellulose.

According to some embodiments, the present invention provides an extrudate comprising between 35% and 70% by weight of a yeast material. According to some embodiments, the present invention provides an extrudate comprising between 35% and 70% by dry weight of a yeast material. In some embodiments, the present invention provides a high moisture extrudate comprising between 35% and 75% by weight of a yeast material. In some embodiments, the present invention provides a high moisture extrudate comprising between 35% and 75% by dry weight of a yeast material. In some embodiments, a high moisture extrudate comprises between 35% and 75% (w/w), 39% and 75% (w/w), 40% and 75% (w/w), 45% and 75% (w/w), 50% and 75% (w/w), 55% and 75% (w/w), 30% and 70% (w/w), 35% and 70% (w/w), 39% and 70% (w/w), 40% and 70% (w/w), 45% and 70% (w/w), 50% and 70% (w/w), 55% and 70% (w/w), 30% and 60% (w/w), 35% and 60% (w/w), 39% and 60% (w/w), 40% and 60% (w/w), 45% and 60% (w/w), 50% and 60% (w/w), 30% and 55% (w/w), 35% and 55% (w/w), 39% and 55% (w/w), 40% and 55% (w/w), or between 45% and 55% (w/w), by dry weight of a yeast material.

In some embodiments, there is provided an extrudate comprising between 35% and 75% by dry weight of a yeast material, and between 30% and 75% (w/w), of an additional protein as described hereinabove.

In some embodiments, the extrudate comprises: between 30% and 75% (w/w) of a yeast material, between 30% and 75% (w/w) of an additional protein, and between 30% and 75% (w/w) of water.

In some embodiments, there is provided a high moisture extrudate comprising between 33% and 75% (w/w) of a yeast material. In some embodiments, there is provided a high moisture extrudate comprising between 35% and 75% by dry weight of a yeast material, and between 30% and 75% (w/w), of an additional protein. In some embodiments, the high moisture extrudate comprises: between 35% and 75% by dry weight of a yeast material, between 30% and 75% (w/w), of an additional protein, and between 30% and 75% (w/w) of water.

In some embodiments, the extrudate is in the form of a fiber. In some embodiments, the high moisture extrudate is in the form of a fiber.

In some embodiments, the extrudate is characterized by a fibrous texture. In some embodiments, the high moisture extrudate is characterized by a fibrous texture.

In some embodiments, the extrudate comprising between 35% and 75% by dry weight of a yeast material, between 30% and 75% (w/w), of an additional protein, and between 30% and 75% (w/w) of water, is in the form of a fiber. In some embodiments, the extrudate comprising between 35% and 75% by dry weight of a yeast material, between 30% and 75% (w/w), of an additional protein, and between 30% and 75% (w/w) of water, is characterized by a fibrous texture.

In some embodiments, the extrudate comprising between 35% and 75% (w/w) of a yeast material, as described herein above, comprises a protein content between 15% and 65%, 18% and 65%, 20% and 65%, 25% and 65%, 30% and 65%, 15% and 60%, 18% and 60%, 20% and 60%, 25% and 60%, 30% and 60%, 15% and 50%, 18% and 50%, 20% and 50%, 25% and 50%, or between 30% and 50%, by weight of the yeast material, including any range therebetween. Each possibility represents a separate embodiment of the invention. In some embodiments, the protein content is calculated by conversion of the nitrogen content. In some embodiments, the protein content is calculated by using 6.25 nitrogen-protein conversion coefficient.

As used herein, “fibrous” refers to any material containing, consisting of, or resembling fibers. In some embodiments, a fibrous material is capable of being separated into fibers. A “fiber” as used herein, is meant a fine cord of fibrous material composed of two or more filaments twisted together. By “filament” is meant a slender, elongated, threadlike object or structure of indefinite length, ranging from microscopic length to lengths of a mile or greater.

In some embodiments, the extrudate comprises between 30% and 75% (w/w), 50% and 75% (w/w), 60% and 75% (w/w), 30% and 70% (w/w), 50% and 70% (w/w), 55% and 70% (w/w), 60% and 70% (w/w), 50% and 69% (w/w), 55% and 69% (w/w), 60% and 69% (w/w), 50% and 65% (w/w), or between 55% and 65% (w/w) of water, including any range therebetween. Each possibility represents a separate embodiment of the invention.

In some embodiments, the extrudate further comprises between 1% and 25% (w/w) of a starch, flour, or both, as described hereinabove. In some embodiments, the extrudate further comprises between 1% and 25% (w/w), 1% and 15% (w/w), 1% and 10% (w/w), 1% and 5% (w/w), 5% and 25% (w/w), 5% and 15% (w/w), or between 5% and 10% (w/w) of a starch, flour, or both.

In some embodiments, the extrudate further comprises between 1% and 25% of a fiber, as described hereinabove. In some embodiments, the extrudate further comprises between 1% and 25% (w/w), 1% and 15% (w/w), 1% and 10% (w/w), 1% and 5% (w/w), 5% and 25% (w/w), 5% and 15% (w/w), or between 5% and 10% (w/w) of a fiber.

In some embodiments, the extrudate comprises between 0.01% and 1.5% (w/w), 0.05% and 1.5% (w/w), 0.09% and 1.5% (w/w), 0.1% and 1.5% (w/w), 0.1% and 0.9% (w/w), 0.2% and 0.9% (w/w), 0.3% and 0.9% (w/w), 0.5% and 0.9% (w/w), 0.1% and 0.7% (w/w), 0.2% and 0.7% (w/w), 0.3% and 0.7% (w/w), 0.5% and 0.7% (w/w), 0.01% and 0.5% (w/w), 0.05% and 0.5% (w/w), 0.09% and 0.5% (w/w), 0.1% and 0.5% (w/w), 0.1% and 0.5% (w/w), 0.2% and 0.5% (w/w), 0.3% and 0.5% (w/w), or between 0.5% and 0.9% (w/w), of an expansion regulating agent, including any range therebetween. Each possibility represents a separate embodiment of the invention. As used herein, “expansion regulating agent” refers to any substance capable of providing a homogeneous extrudate structure. In some embodiments, the expansion regulating agent is capable of providing an expanded extrudate with a well-defined structure. In some embodiments, an expansion regulating agent regulates the pore size by increasing the uniformity of the pores and by forming smaller pores. In some embodiments, an expansion regulating agent comprises sodium salts, calcium salts, magnesium salts or others.

In some embodiments, the expansion regulating agent comprises calcium carbonate. In some embodiments, the extrudate comprises between 0.01% and 1.5% (w/w), 0.05% and 1.5% (w/w), 0.09% and 1.5% (w/w), 0.1% and 1.5% (w/w), 0.1% and 0.9% (w/w), 0.2% and 0.9% (w/w), 0.3% and 0.9% (w/w), 0.5% and 0.9% (w/w), 0.1% and 0.7% (w/w), 0.2% and 0.7% (w/w), 0.3% and 0.7% (w/w), 0.5% and 0.7% (w/w), 0.01% and 0.5% (w/w), 0.05% and 0.5% (w/w), 0.09% and 0.5% (w/w), 0.1% and 0.5% (w/w), 0.1% and 0.5% (w/w), 0.2% and 0.5% (w/w), 0.3% and 0.5% (w/w), or between 0.5% and 0.9% (w/w) of calcium carbonate, including any range therebetween. Each possibility represents a separate embodiment of the invention.

In some embodiments, the extrudate further comprises a coloring agent, flavoring agent, flavor masking agent, or any combination thereof. Non-limiting examples of thickening agents according to the present invention include starch rich ingredients such as corn flour, tapioca flour, Alginate, beans or bean extract. Non-limiting examples of emulsifiers according to the present invention include aquafaba, coconut milk, pea protein, lecithin, quinoa protein and other proteins. In some embodiments, the extrudate further comprises a vegetal fat.

In some embodiments, the yeast material is derived from downstream food-related industries. In some embodiments, the yeast material is derived from beer downstream. In some embodiments, the yeast material is derived from yeast extract downstream.

In some embodiments, the yeast comprises Saccharomycescerevisiae, Streptomycesnatalensis, Streptomyceschattanoogensis, Saccharomycesfragilis, Candidautilis, Candidaguilliermondii, Candidalipolityca, Cyberlindnerajadinii, or any combination thereof. Each possibility represents a separate embodiment of the invention.

As used herein the term “yeast” refers to eukaryotic, single-celled microorganisms from the phyla Ascomycota and Basidiomycota. An exemplary yeast is budding yeast from the order Saccharomycetales. A particular example of yeast is Saccharomyces spp., including but not limited to Saccharomycescerevisiae.

As used herein, “material” refers to the large source of carbon-based compounds found within natural and engineered, terrestrial and aquatic environments. In some embodiments, “material” refers to any matter composed of organic compounds that have come from organisms such as plants, animals and microorganisms, or their remains.

The term “yeast material” as used herein, refers to any compound constituting, secreted, derived or produced by yeast. In some embodiments, yeast material is a carbon-based material.

In some embodiments, the yeast material comprises: a whole yeast, a yeast extract, a yeast biomass, a yeast homogenate, a yeast filtrate, a yeast concentrate, any fraction thereof, or any combination thereof. In some embodiments, the yeast material is selected from the group consisting of: a whole yeast, a yeast biomass, a yeast filtrate, a yeast concentrate, any fraction thereof, and any combination thereof. In some embodiments, the yeast material comprises or consists of a whole yeast. In some embodiments, the yeast material is devoid of a yeast extract.

The term “extrudate” as used herein, refers to a product in which the composition is heated and/or compressed to a molten (or softened) state and subsequently extruded. As used herein, the term “extrudable composition” refers to the ability of a composition to be extruded.

### Food Product

According to some embodiments, the present invention provides a food product comprising an extrudate as described hereinabove. In some embodiments, there is provided a food product consisting essentially of an extrudate as described hereinabove.

In some embodiments, the present invention provides a food product comprising between 35% and 100% by dry weight of a yeast material. In some embodiments, the present invention provides a food product comprising between 35% and 99%, 50% and 99%, between 55% and 99%, between 60% and 99%, between 65% and 99%, 50% and 95%, between 55% and 95%, between 60% and 95%, between 65% and 95%, 50% and 90%, between 55% and 90%, between 60% and 90%, between 65% and 90%, between 50% and 70%, between 55% and 70%, between 60% and 70%, between 65% and 70%, 50% and 69%, between 55% and 69%, between 60% and 69%, between 65% and 69%, 50% and 65%, or between 55% and 65%, by dry weight of a yeast material including any range therebetween. Each possibility represents a separate embodiment of the invention.

In some embodiments, the present invention provides a food product comprising between 30% and 99%, 50% and 99%, between 55% and 99%, between 60% and 99%, between 65% and 99%, 50% and 95%, between 55% and 95%, between 60% and 95%, between 65% and 95%, 50% and 90%, between 55% and 90%, between 60% and 90%, between 65% and 90%, between 50% and 70%, between 55% and 70%, between 60% and 70%, between 65% and 70%, 50% and 69%, between 55% and 69%, between 60% and 69%, between 65% and 69%, 50% and 65%, or between 55% and 65%, by weight of the yeast material, including any range therebetween. Each possibility represents a separate embodiment of the invention. In some embodiments, the yeast material is selected from the group consisting of: a whole yeast, a yeast biomass, a yeast filtrate, a yeast concentrate, a yeast extrudate, any fraction thereof, and any combination thereof. In some embodiments, the yeast material comprises a yeast protein extract.

In some embodiments, the food product, comprises a protein content between 10% and 90%, 12% and 90%, 15% and 90%, 20% and 90%, 50% and 90%, 50% and 90%, 10% and 85%, 12% and 85%, 15% and 85%, 20% and 85%, 50% and 85%, 50% and 85%, 10% and 80%, 12% and 80%, 15% and 80%, 20% and 80%, 50% and 80%, 50% and 80%, 10% and 75%, 12% and 75%, 15% and 75%, 20% and 75%, 50% and 75%, or between 50% and 75%, by weight of the yeast material, including any range therebetween. Each possibility represents a separate embodiment of the invention.

In some embodiments, the food product comprises a nitrogen content between 1% and 15%, 4% and 15%, 5% and 15%, 6% and 15%, 7% and 15%, 1% and 10%, 4% and 10%, 5% and 10%, 6% and 10%, 7% and 10%, 4% and 7%, or between 5% and 7%, by weight of the yeast material, including any range therebetween. Each possibility represents a separate embodiment of the invention. In some embodiments, the protein content is calculated by conversion of the nitrogen content. In some embodiments, the protein content is calculated by using 6.25 nitrogen-protein conversion coefficient.

In some embodiments, the food product is a meat-alternative food product. In some embodiments, the food product is a dairy-alternative food product. In some embodiments, the food product is an egg-alternative food product. In some embodiments, the food product is a vegetarian food product. In some embodiments, the food product is a vegan food product.

As used herein, the term “food product” refers to a material, a substance, or an additive, which can be used as food, or which can be added to food. Typically, the food product is any composition that an animal, preferably a mammal such as a human, may consume as part of its diet.

As used herein, “meat-alternative” refers to meat analogs comprising high content of protein, capable of being consumed as such. In some embodiments, meat-alternative is a plant-based meat alternative.

In some embodiments, a meat-alternative, dairy alternative or egg alternative-food product according to the present invention, is characterized by similar properties of a correspondent meat food product, dairy food product or egg food product. Properties of meat eggs and dairy that can be tested include mechanical properties such as hardness, cohesiveness, brittleness, chewiness, gumminess, viscosity, elasticity, and adhesiveness. Properties that can be tested also include geometric properties such as particle size and shape, and particle shape and orientation. Additional properties can include moisture content and fat content. These properties can be described using terms such as “soft,” “firm” or “hard” describe hardness; “crumbly,” “crunchy,” “brittle,” “chewy,” “tender,” “tough,” “short,” “mealy,” “pasty,” or “gummy,” to describe cohesiveness; “thin” or “viscous” to describe viscosity; “plastic” or “elastic” to describe elasticity; “sticky,” “tacky” or “gooey” to describe adhesiveness; “gritty,” “grainy” or “course” to describe particle shape and size; “fibrous,” “cellular” or “crystalline” to describe particle shape and orientation, “dry,” “moist,” “wet,” or “watery” to describe moisture content; or “oily” or “greasy” to describe fat content.

In some embodiments, a test group of people can be asked to rate a certain meat, egg or dairy product, according to properties which describe the product and compare it to a food product according to the present invention. These ratings can be used as an indication of the properties of the product. In some embodiments, the food products of the present invention are rated similar to meat, eggs or dairy, according to human evaluation. In some embodiments the food product is indistinguishable from real meat to a human. In some embodiments the food product is indistinguishable from dairy to a human. In some embodiments the food product is indistinguishable from eggs meat to a human.

As used herein, “vegetarian” refers to properties of the components, and indicates that the components are not sourced from or derived from meat, poultry, game, fish, shellfish or by-products of animal slaughter.

In some embodiments, the present invention provides a food product prepared using the extrudates or yeast materials described herein. In some embodiments, the food product prepared with the extrudates or yeast materials described herein, is indistinguishable from an equivalent food product prepared with meat, eggs, dairy products, or any combination thereof.

In some embodiments, the food product is characterized as being suitable for use as an equivalent product to meat, eggs, dairy products, or any combination thereof.

In some embodiments, the food product prepared with the extrudates or yeast materials described herein, is characterized as an equivalent product prepared using meat, eggs, dairy products, or any combination thereof. In some embodiments, equivalent product refers to any one of texture, color, and flavor.

In some embodiments, the food product prepared with the extrudates or yeast materials described herein, has at least 80%, at least 85%, at least 90%, or at least 95% of the characteristics and sensory properties of a food product prepared using meat, eggs, dairy products, or any combination thereof.

In some embodiments, the food product is characterized by at least 80%, at least 85%, at least 90% of the texture, color, and flavor of an equivalent product prepared using meat, eggs, dairy products, or any combination thereof. In some embodiments, the food product is characterized by at least 60% of the odor profile of an equivalent product prepared using meat, eggs, dairy products, or any combination thereof. In some embodiments, the food product is characterized by at least 65%, at least 70%, at least 75%, at least 80%, at least 85%, at least 90%, or at least 95%, of the odor profile of an equivalent product prepared using meat, eggs, dairy products, or any combination thereof. In some embodiments, the food product is characterized by at least 60% of the consistency of an equivalent product prepared using eggs. In some embodiments, the food product is characterized by at least 60%, at least 65%, at least 70%, at least 75%, at least 80%, at least 85%, at least 90%, or at least 95%, of the consistency of an equivalent product prepared using meat, eggs, dairy products, or any combination thereof, including any value therebetween. Each possibility represents a separate embodiment of the invention.

In some embodiments, the food product is devoid of meat, eggs, dairy products, or any combination thereof. In one embodiment, “free of” is “devoid of” or essentially “devoid of”.

As used herein, the terms “egg” “meat”, “dairy products”, for example, as used when describing a “product devoid of meat, eggs, dairy products,” refers to an animal product or any component of an animal product.

As used herein, the term “vegan” refers to properties of the components, and indicates that the components are not sourced from or derived from an animal or animal product. As such, the components that are “vegan” are free of any animal products or animal byproducts. What constitutes an animal product or byproduct is well known in this field, and to those following a vegetarian or vegan diet. In particular, the term “animal product” refers to any animal parts, animal byproducts, or products produced by an animal. Some examples of materials that would be considered “animal products” include those parts of the animal that are consumable or typically prepared for consumption by humans (including, e.g., fat, flesh, blood, etc.). Products produced by an animal are also considered “animal products” as used herein, and refer to the products produced by an animal without slaughtering the animal, (e.g., milk, eggs, honey, etc.). “Animal byproducts” are products that are typically not consumable by themselves but are the byproducts of slaughtering animals for consumption, e.g., bones, carcasses, etc. However, animal byproducts are often processed into human consumable foodstuffs, some well-known examples of which include gelatin, casein, whey, rennet, etc. As used herein, these processed animal byproducts (e.g., gelatin, casein, whey, rennet, etc.) are encompassed by the term “animal byproducts.” As described herein, “vegan” and “plant-based” components or ingredients are substantially free (or in some embodiments, completely free) of such animal products and byproducts.

In some embodiments, compositions and food products as described herein are suitable for a vegan diet and/or a vegetarian diet. For example, in embodiments in which the composition is suitable for a vegan diet, the composition may include primarily plant-based components such that the composition contains substantially no animal products, animal byproducts, or substantially no components derived from these animal sources.

In some embodiments, the food product is in the form of a nugget, a breast, a steak, or a minced meat-like product.

In some embodiments, the food product is in the form of a sushi or fish substitute, or cheese substitute. In some embodiments, the food product is devoid of soy.

### Texture

According to some embodiments, there is provided a method to modify or improve the texture of the yeast and/or yeast compositions and extrudates as an animal product substitute as described hereinabove.

In some embodiments, texture is improved by physical processes. A non-limiting example includes mechanical manipulation, to modify the texture of the product containing yeast: the temperature of a mixture of yeast in a solvent such as water can be decreased to the point where the solution freezes. At such a temperature, crystals are formed that can break and distort the structure of the yeast cell walls in such a way that it may render the mechanical properties of the yeast to change. Once this process has occurred, the yeast or yeast solution can be unfrozen and the yeast can continue the process towards becoming an ingredient/base for a food product. Another non-limiting example includes processing similar to textured vegetal protein (TVP) with dry, hot or wet extrusion. In dry or hot extrusion, yeast or yeast flour or concentrate or extract can be extruded into various shapes (chunks, flakes, nuggets, grains, and strips) and sizes. In typical extrusion, the defatted thermoplastic proteins are heated to 150-200° C. high pressure for example 80 to 130 bar, (which denatures them into a fibrous, insoluble, porous network that can soak up as much as three times its weight in liquids). In high-moisture extrusion, the water temperature typically ranges between 140-165° C. and the pressurized molten protein mixture exits the extruder with a water content of typically approximately 70%.

In some embodiments, the texture is improved by mixing the yeast with vegetal fibers (i.e. cellulose, mushroom, extruded protein, gluten, or other), or other vegetal ingredients with high fiber and/or high protein content, for instance oat flakes. In some embodiments, the vegetal ingredients are in the form of flakes. Non-limiting examples of high-fiber vegetal ingredients include oat, wheat germs, cotton seeds, outer husk or bran of cereals, barley, different sorts of peas, beans, lentils, artichoke, corn and other cereal post-harvest by-products, wheatgrass, other grass-like vegetal ingredients, beetroot, carrot, apple or the like.

In some embodiments calcium or magnesium, or other salts are added to the compositions to cause coagulation of a yeast-containing solution or mixture. In some embodiments, physical pressure is applied to yeast-containing solution or mixture in a mold to provide shape and texture.

### Coloring Agents

In some embodiments, a food product or composition as described herein, comprises one or more coloring agents. Various natural or artificial coloring agents are known to those skilled in the art, and can include, for example, carotenoids such as beta-carotene, turmeric, annatto, mango yellow, or palm-based oils. In some embodiments, a food product or composition as described herein, comprises 0.1% to 20% (w/w), 0.5% to 15% (w/w), 0.3% to 15% (w/w), 0.4% to 15% (w/w), 0.5% to 15% (w/w), 0.4% to 10% (w/w), 0.5% to 5% (w/w), or 0.4% to 2% (w/w) of a coloring agent, including any range therebetween. Each possibility represents a separate embodiment of the invention.

### Flavoring Agents

In some embodiments, a food product or composition as described herein, comprises one or more flavoring agents. In some embodiments, a food product or composition as described herein, comprises yeast, sugar, salt, and any combination thereof. Various natural or artificial flavoring agents are known to those skilled in the art, and can include, for example, salt, spices, sugar, sweeteners, monosodium glutamate, sulfuric flavoring agents such as black salt, or other flavoring agents. In some embodiments, the a food product or composition as described herein, comprises 0.1% to 20% (w/w), 0.5% to 15% (w/w), 0.3% to 15% (w/w), 0.4% to 15% (w/w), 0.5% to 15% (w/w), 0.4% to 10% (w/w), 0.5% to 5% (w/w), or 0.4% to 2% (w/w) of a flavoring agent, including any range therebetween. Each possibility represents a separate embodiment of the invention.

### Other Agents

In some embodiments, a food product or composition as described herein, further comprises an emulsifier. In some embodiments, the composition comprises 0.1% to 5% (w/w), 0.2% to 5% (w/w), 0.3% to 5% (w/w), 0.4% to 5% (w/w), 0.5% to 5% (w/w), 0.5% to 4% (w/w), or 0.5% to 3% (w/w), of an emulsifier, including any range therebetween. Each possibility represents a separate embodiment of the invention.

In some embodiments, a food product or composition as described herein, comprises a thickener. 0.1% to 5% (w/w), 0.2% to 5% (w/w), 0.3% to 5% (w/w), 0.4% to 5% (w/w), 0.5% to 5% (w/w), 0.5% to 4% (w/w), or 0.5% to 3% (w/w), of a thickener, including any range therebetween.

In some embodiments, a food product or composition as described herein, comprises 0.5% to 15% (w/w), 1% to 15% (w/w), 0.5% to 12% (w/w), 0.5% to 10% (w/w), or 1% to 10% (w/w) of an oil, including any range therebetween. Each possibility represents a separate embodiment of the invention.

In some embodiments, the oil is a vegetable-based oil. Examples of vegetable oils that may be used according to the present invention include, but are not limited to, soybean oil, safflower oil, linseed oil, corn oil, sunflower oil, olive oil, canola oil, sesame oil, cottonseed oil, palm oil, rapeseed oil, tung oil, or a blend of any of these oils. Alternatively, any partially hydrogenated vegetable oils or genetically modified vegetable oils can be used. Examples of partially hydrogenated vegetable oils or genetically modified vegetable oils include, but are not limited to, high oleic safflower oil, high oleic soybean oil, high oleic peanut oil, high oleic sunflower oil and high erucic rapeseed oil (crambe oil).

### Improving Yeast Taste and Flavor

According to some embodiments, there is provided a method for improving the flavor of a yeast material, comprising contacting the yeast material with an agent, wherein the agent is characterized by being able to adjust the pH of the yeast material to a pH ranging from 4 to 7.5, 4.5 to 7.5, 4.9 to 7.5, 5 to 7.5, 5.5 to 7.5, 4 to 7, 4.5 to 7, 4.9 to 7, 5 to 7, 5.5 to 7, 4 to 6.5, 4.5 to 6.5, 4.9 to 6.5, 5 to 6.5, or 5.5 to 6.5, including any range therebetween. Each possibility represents a separate embodiment of the invention.

In some embodiments, improving comprises reducing yeast taste, off-flavor of yeast taste, enhance meat flavor, or any combination thereof.

In some embodiments, there is provided a method for improving the bitterness of a yeast material as described herein.

In some embodiments, the agent is a naturally occurring substance suitable to be used as an alkali material in solution. Non-limiting examples of natural vegetal sources that can be used as an alkali solution/material / are asparagus, lettuce, almond, arugula, amaranth, avocado or avocado oil, beetroot, broccoli sprout, cabbage, buckwheat, basil, carrot, cauliflower, celery, chia, Chives, Cilantro, coconut, collard, cucumber, cumin, eggplant, endive, flax oil and seeds, ginger, beans, kale, leeks, kelp, lentils, millet, mustard greens, potato, okra, parsley, peas, peppers, pumpkins and its seeds, quinoa, radish, spinach, squash, sunflower seeds, sweet potato, zucchini, wheatgrass, watercress, and other leaves. In some embodiments, the natural alkali source (whole parts of a vegetal, or extract or solution) as described herein, is used either as part of the formulation of the end-product incorporating yeast or used to produce a solution or solution like liquid / fluid or powder used to change the yeast pH as part of the end-product production process.

In some embodiments, the method further comprises a step of separating the yeast from the agent. The separation can be performed by any means known in the art. In some embodiments separation is performed by centrifugation.

In some embodiments, masking agents are added to the formulations. As used herein, masking agent refers to an agent capable of counteract/mask the bitter flavors, or agents that block totally or partially the bitterness receptors on human/animal tongues.

In some embodiments, the masking agent is a natural masking agent. Non-limiting examples of natural masking agents include ginger, onion, mushroom/ fungi, garlic, any extract or combination thereof.

In some embodiments, ion-exchange separation methods are used to at least partially debitter the yeast or improve its flavor and taste. In some embodiments, ion-exchange methods include resins. The use of resin typically requires the yeast to be diluted into a liquid. Resins are for example used as beads in columns. A non-limiting examples of suitable resins include DOWEX OPTIPORE™ or a resin presenting similar physical and/ or chemical properties. The ion-exchange separation technique is optionally used with activated carbon and optionally uses cation or anion charges.

In some embodiments, the method comprises the step of mechanical grinding of dry yeast and/or vacuum and/or heating, to remove volatile compounds from the yeast.

### General

As used herein the term “about” refers to ± 10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### High Moisture Extrudates and Low Moisture Extrudates

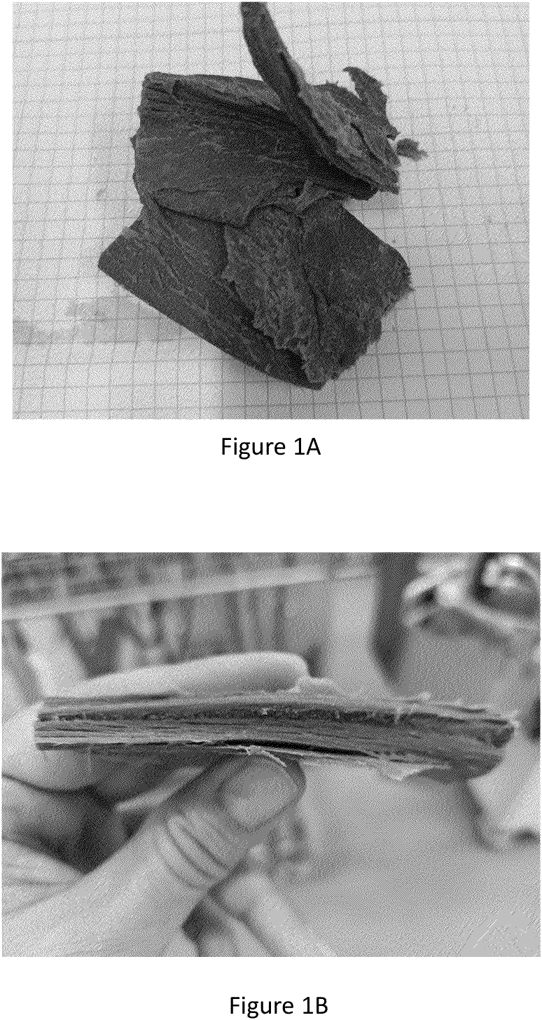

High moisture extrudates (HME) were successfully obtained using yeast as main ingredient (Table 1). FIGS. 1A-B present pictures of the high moisture extrudate 3 (HME3) described in Table 1. It can be observed that the extrudate presents a fiber-like structure.

TABLE 1

Formulation of HME3

| INGREDIENTS | %DM in recipe | % in recipe | % Protein content | % Protein in recipe | % Fat | % Fat in recipe |
|---|---|---|---|---|---|---|
| Water | 0.0% | 52.0% | 0% | 0% | 0.0% | 0.0% |
| Yeast (SAF Instant RED) | 40.2% | 19.3% | 24% | 5% | 5.7% | 1.1% |
| Pumpkin seed protein | 54.2% | 26.0% | 65% | 17% | 9.0% | 2.3% |
| Vegetable oil | 4.2% | 2.0% | 0% | 0% | 100.0% | 2.0% |
| Calcium carbonate | 1.5% | 0.7% | 0% | 0% | 0.0% | 0.0% |
| TOTAL | 100.0% | 100.0% | | 22% | | 5.4% |

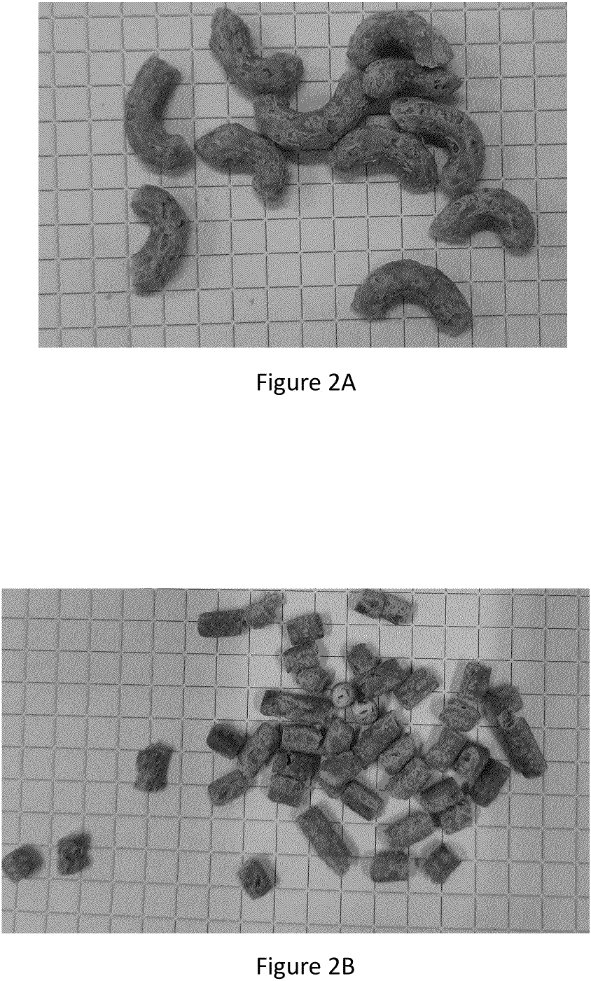

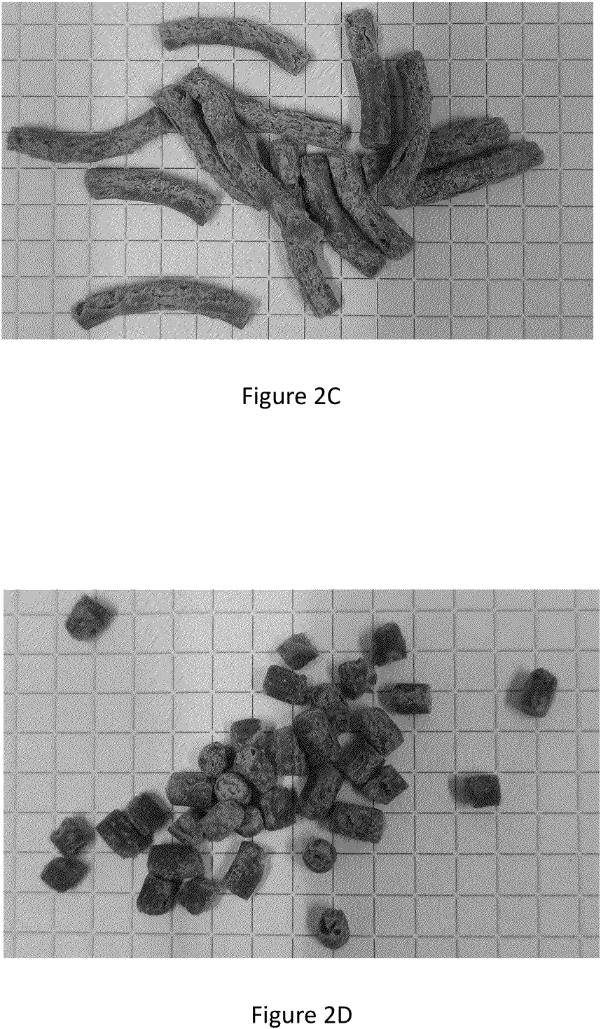

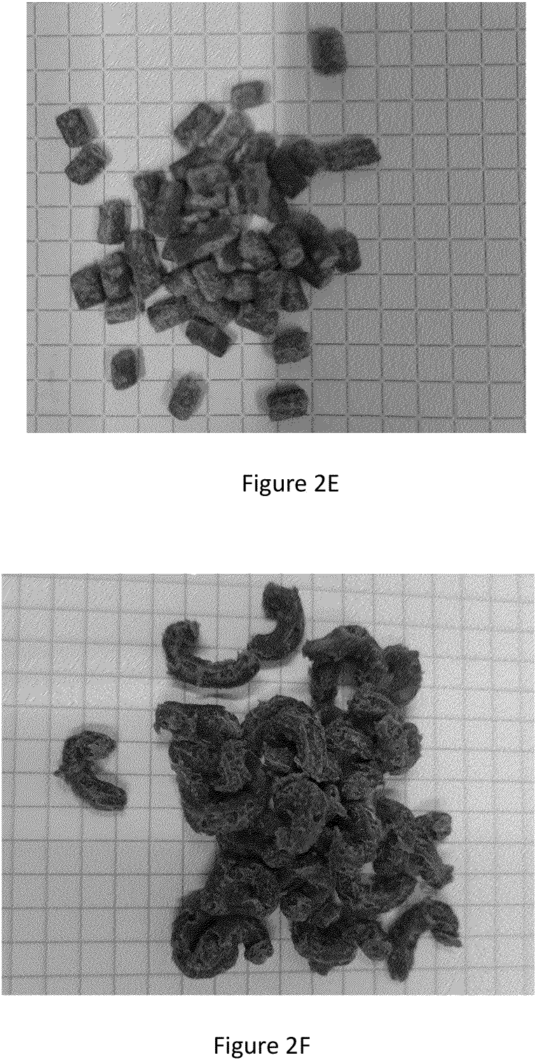

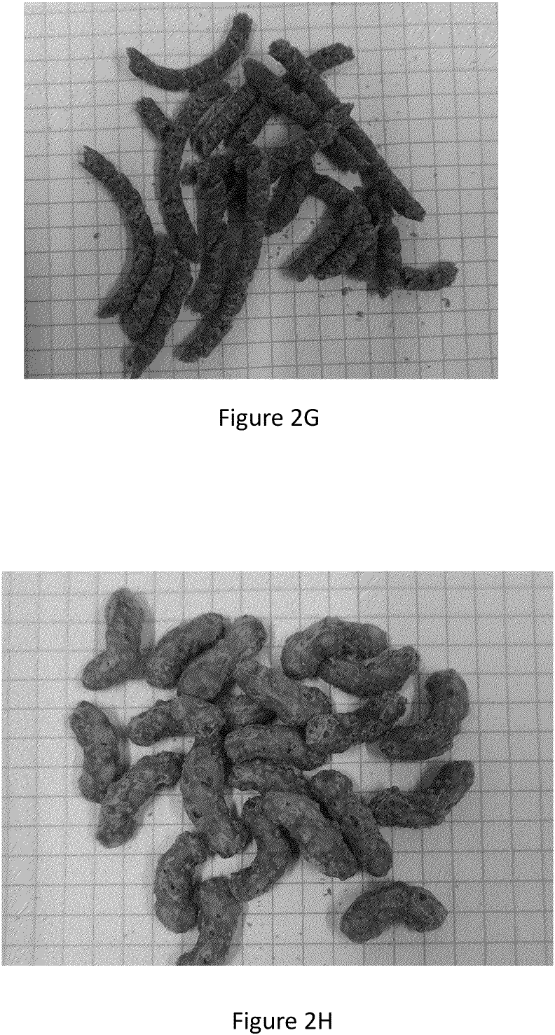

Yeast was also used as main ingredient in low moisture extrudates (LME). Several extrudates were successfully obtained (FIGS. 2A-H) from different formulations (Tables 2-9).

TABLE 2

Formulation of LME1

| INGREDIENTS | %DM in recipe | % in recipe | % Protein content | % Protein in recipe |
|---|---|---|---|---|
| Water | 0.0% | 9.5% | 0% | 0% |
| Yeast (SAF Instant Red) | 77.3% | 70.0% | 40% | 28% |
| Corn grits | 22.1% | 20.0% | 0% | 0% |
| Calcium Carbonate | 0.6% | 0.5% | 0% | 0% |
| TOTAL | 100.0% | 100.0% | | 28% |

TABLE 3

Formulation of LME2

| INGREDIENTS | %DM in recipe | % in recipe | % Protein content | % Protein in recipe |
|---|---|---|---|---|
| Water | 0.0% | 9.5% | 0% | 0% |
| Yeast (SAF Instant RED) | 99.4% | 90.0% | 40% | 36% |
| Calcium Carbonate | 0.6% | 0.5% | 0% | 0% |
| TOTAL | 100.0% | 100.0% | | 36% |

TABLE 4

Formulation of LME3

| INGREDIENTS | %DM in recipe | % in recipe | % Protein content | % Protein in recipe |
|---|---|---|---|---|
| Water | 0.0% | 9.5% | 0% | 0% |
| Nutritional yeast BA13 (50% protein) | 99.4% | 90.0% | 50% | 45% |
| Calcium Carbonate | 0.6% | 0.5% | 0% | 0% |
| TOTAL | 100.0% | 100.0% | | 45% |

TABLE 5

Formulation of LME4

| INGREDIENTS | %DM in recipe | % in recipe | % Protein content | % Protein in recipe |
|---|---|---|---|---|
| Water | 0.0% | 9.5% | 0% | 0% |
| Yeast extract (Bastex) | 44.2% | 40.0% | 24% | 10% |
| Yeast (SAF Instant RED) | 55.2% | 50.0% | 40% | 20% |
| Calcium Carbonate | 0.6% | 0.5% | 0% | 0% |
| TOTAL | 100.0% | 100.0% | | 30% |

TABLE 6

Formulation of LME5

| INGREDIENTS | %DM in recipe | % in recipe | % Protein content | % Protein in recipe |
|---|---|---|---|---|
| Water | 0.0% | 9.5% | 0% | 0% |
| Yeast (Lallemand Inactive dry Yeast Co.) 2130 | 44.2% | 40.0% | 40% | 16% |
| Yeast (SAF Instant RED) | 55.2% | 50.0% | 40% | 20% |
| Calcium Carbonate | 0.6% | 0.5% | 0% | 0% |
| TOTAL | 100.0% | 100.0% | | 36% |

TABLE 7

Formulation of LME6

| INGREDIENTS | %DM in recipe | % in recipe | % Protein content | % Protein in recipe |
|---|---|---|---|---|
| Water | 0.0% | 8.7% | 0% | 0% |
| Yeast (Lallemand Inactive dry Yeast Co.) 2255 | 43.8% | 40.0% | 40% | 16% |
| Yeast (SAF Instant RED) | 54.8% | 50.0% | 40% | 20% |
| Flavor Maxarome DSM | 0.2% | 0.2% | 0% | 0% |
| Masking Agent | 0.7% | 0.6% | 0% | 0% |
| Calcium Carbonate | 0.5% | 0.5% | 0% | 0% |
| TOTAL | 100.0% | 100.0% | | 36% |

TABLE 8

Formulation of LME9

| INGREDIENTS | %DM in recipe | % in recipe | % Protein content | % Protein in recipe |
|---|---|---|---|---|
| Water | 0.0% | 9.5% | 0% | 0% |
| Nutritional yeast BA13 (50% protein) | 77.3% | 70.0% | 50% | 35% |
| Corn grits | 22.1% | 20.0% | 0% | 0% |
| Calcium Carbonate | 0.6% | 0.5% | 0% | 0% |
| TOTAL | 100.0% | 100.0% | | 35% |

TABLE 9

Formulation of LME12

| INGREDIENTS | %DM in recipe | % in recipe | % Protein content | % Protein in recipe |
|---|---|---|---|---|
| Water | 0.0% | 9.5% | 0% | 0% |
| Soy protein (Alpha 8 67%) | 66.3% | 60.0% | 67% | 40% |
| Nutritional yeast BA13 (50% protein) | 33.1% | 30.0% | 50% | 15% |
| Calcium Carbonate | 0.6% | 0.5% | 0% | 0% |
| TOTAL | 100.0% | 100.0% | | 55% |

### Example 2

### pH Modulation Experiments

An experiment was performed in order to adjust the taste of yeast material by modulating the pH of a solution (Table 10). 2 grams (g) of yeast were mixed with 100 g of water, at room temperature. To adjust alkaline pH baking soda was used, and to adjust acidic pH 5% vinegar was used.

TABLE 10

| | | | | | |
|---|---|---|---|---|---|
| pH | 5.5 | 6 | 7 | 8 | 8.75 |
| Taste intensity | 4 | 4 | 4 | 7 | 5 |
| Color | light Brown to yellow | light brown to yellow | light brown to yellow | darker brown | darker brown |

With the lowering of the pH, it was observed a reduction in the taste intensity.

Further experiments were performed to verify the results, with different yeast samples: instant yeast, inactive yeast, yeast extract downstream (DS) and spent. The results are presented in Tables 11-12.

2 g of yeast were mixed with 100 g of water, at room temperature. To adjust alkaline pH baking soda was used, and to adjust acidic pH citric acid was used. The different yeast samples were compared for their taste intensity (Table 11), and color (Table 12).

As observed in the previous results (Table 10), it can be observed that in all the yeast sources, the taste intensity decreases with the pH decrease (Table 11).

TABLE 11

Taste intensity results

| | | | | | |
|---|---|---|---|---|---|
| pH | 5 | 6 | 7 | 8 | 9 |
| Instant yeasts | 3 | 3 | 3 | 7 | 9 |
| Inactive yeasts | 3 | 3 | 5 | 8 | 9 |
| Yeasts extract DS | 6 | 5 | 5 | 8 | 9 |
| Spent brewery Yeasts | 6 | 4 | 7 | 10 | 6 |

It can also be observed that in low acidity pH the color is lighter than higher alkali pH (Table 12).

TABLE 12

Color results

| pH | 5 | 6 | 7 | 8 | 9 |
|---|---|---|---|---|---|
| Instant yeasts | 3 | 3 | 3 | 4 | 4 |
| Inactive yeasts | 4 | 4 | 5 | 5 | 4 |
| Yeasts extract DS | 4 | 4 | 5 | 7 | 6 |
| Spent brewery Yeasts | 6 | 8 | 8 | 8 | 8 |

### Example 3

### Yeast Extrudates as Meat Replacement Products

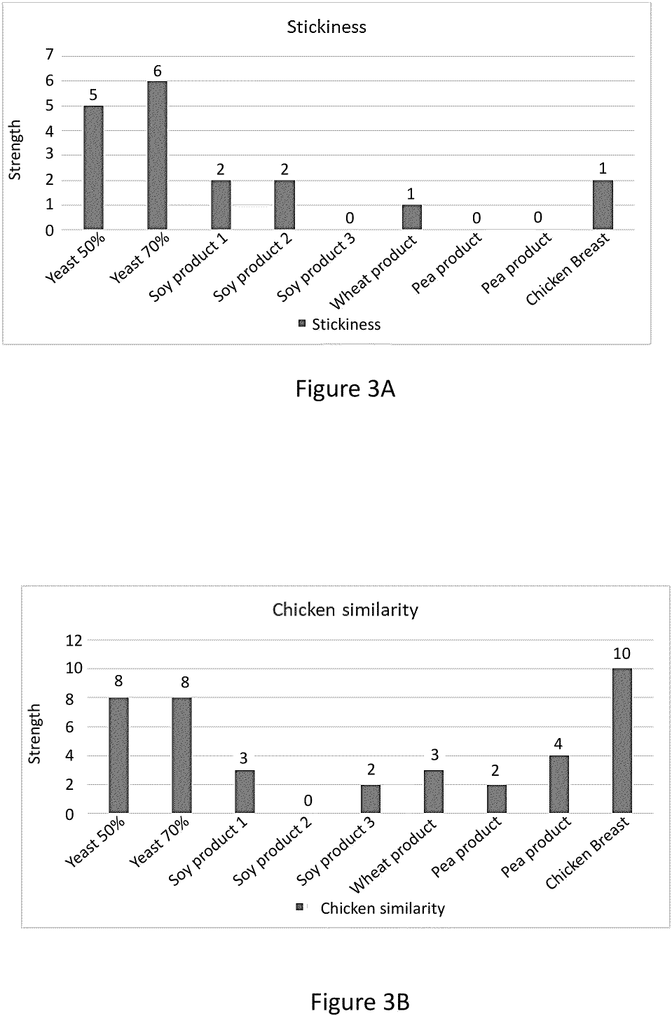

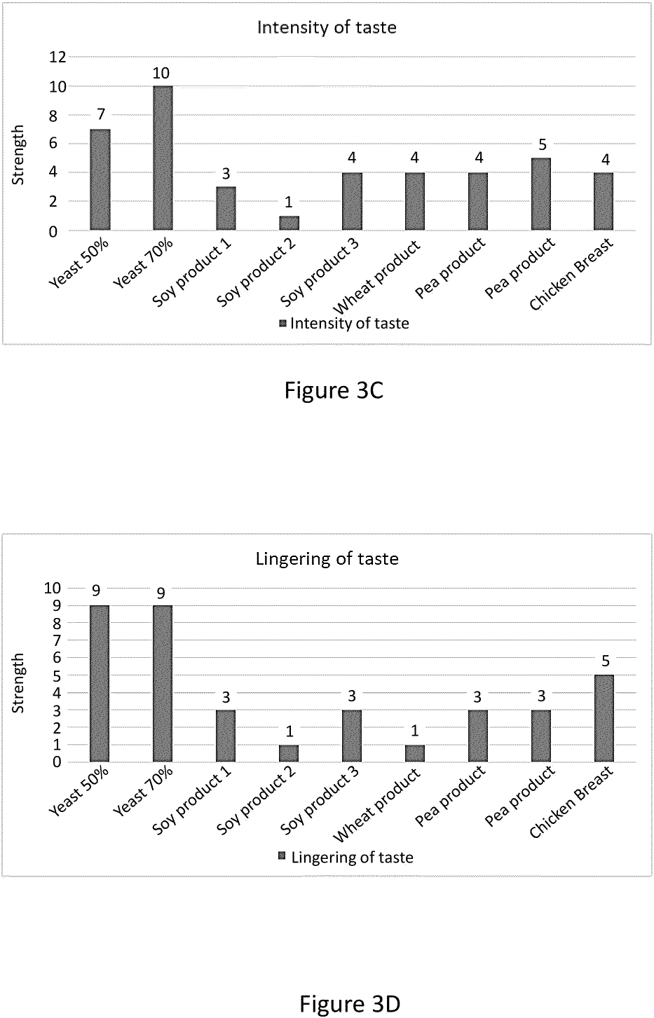

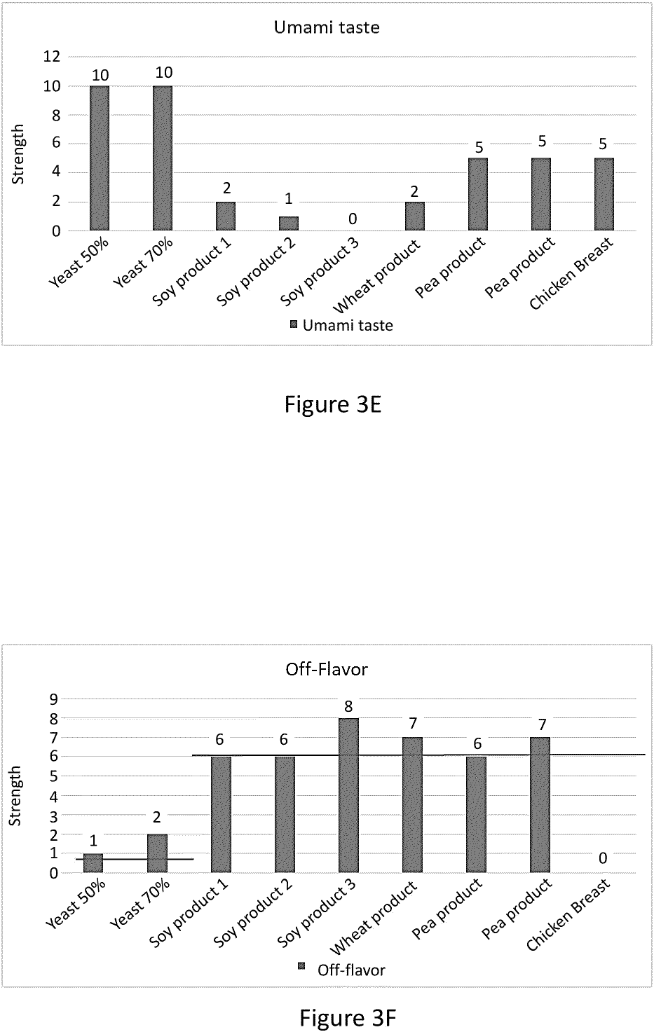

An experiment was performed to assess the sensory properties of a yeast extrudate as compared to Soy, pea, wheat protein extrudate and real chicken (FIGS. 3A-F). Overall, yeast extrudates presented good characteristics comparable to chicken.

Several patty recipes were tested comprising yeast (LME R1, LME R2, LME R3 and LME R9)and compared with patty comprising soy (LME R11) and chicken patty (Patty chicken 6) (Table 13).

TABLE 13

Patty recipes

| Ingredient | Patty with Yeast extrudate 1 | | Patty with Yeast extrudate 2 | | Patty with Yeast extrudate 3 | | Patty with Yeast extrudate 4 | | Patty with Yeast extrudate 5 | | Patty chicken 6 | |
|---|---|---|---|---|---|---|---|---|---|---|---|---|
| Extrudate recipe | LME R1* | | LME R2* | | LME R3* | | LME R9* | | LME R11* | | - | |
| | weight (gr) | % | weight (gr) | % | weight (gr) | % | weight (gr) | % | weight (gr) | % | weig ht (gr) | % |
| Extrudate | 100 | 34 % | 100 | 34% | 100 | 34% | 70 | 27% | 50 | 21% | 0 | 0% |
| water absorption | 125 | 43 % | 125 | 43% | 125 | 43% | 126 | 49% | 125 | 53% | 0 | 0% |
| Chicken breast cubes | 0 | 0% | 0 | 0% | 0 | 0% | 0 | 0% | 0 | 0% | 0 | 0% |
| Minced chicken breast | 0 | 0% | 0 | 0% | 0 | 0% | 0 | 0% | 0 | 0% | 175 | 72% |
| Onion grated | 22.5 | 8% | 22.5 | 8% | 22.5 | 8% | 19.6 | 8% | 17.5 | 7% | 17.5 | 7% |
| Whole eggs | 0.0 | 0% | 0.0 | 0% | 0.0 | 0% | 0.0 | 0% | 0.0 | 0% | 25.0 | 10% |
| Garlic powder | 2.5 | 1% | 2.5 | 1% | 2.5 | 1% | 2.2 | 1% | 1.9 | 1% | 2.2 | 1% |
| Salt | 3.7 | 1% | 3.7 | 1% | 3.7 | 1% | 3.2 | 1% | 2.9 | 1% | 3.3 | 1% |
| Black pepper | 0.6 | 0% | 0.6 | 0% | 0.6 | 0% | 0.5 | 0% | 0.5 | 0% | 0.5 | 0% |
| Paprika | 2.5 | 1% | 2.5 | 1% | 2.5 | 1% | 2.2 | 1% | 1.9 | 1% | 2.2 | 1% |
| Soda powder | 1.2 | 0% | 1.2 | 0% | 1.2 | 0% | 1.1 | 0% | 1.0 | 0% | 1.1 | 0% |
| Oil added | 12.8 | 4% | 12.8 | 4% | 12.8 | 4% | 15.7 | 6% | 20.0 | 8% | 15.8 | 7% |
| Potato protein | 10.6 | 4% | 10.6 | 4% | 10.6 | 4% | 9.4 | 4% | 8.6 | 4% | 0.0 | 0% |
| Potato starch | 7.6 | 3% | 7.6 | 3% | 7.6 | 3% | 6.7 | 3% | 6.2 | 3% | 0.0 | 0% |
| Pregelatin ized potato starch | 2.4 | 1% | 2.4 | 1% | 2.4 | 1% | 2.2 | 1% | 2.0 | 1% | 0.0 | 0% |
| Fried oil Canola | | | | | | | | | | | | |
| Total (gr) | 291.4 | 100 % | 291.4 | 100 % | 291.4 | 100 % | 258.7 | 100 % | 237.4 | 100 % | 242.5 | 100 % |
| * ingredients of the extrudate | Instant yeasts, corn grits, water, calcium carbonate | | Instant yeasts, water, calcium carbonate | | Inactive nutritional yeasts, water, calcium carbonate | | Inactive nutritional, corn grits, water, calcium carbonate | | Soy protein, water, calcium carbonate | | | |

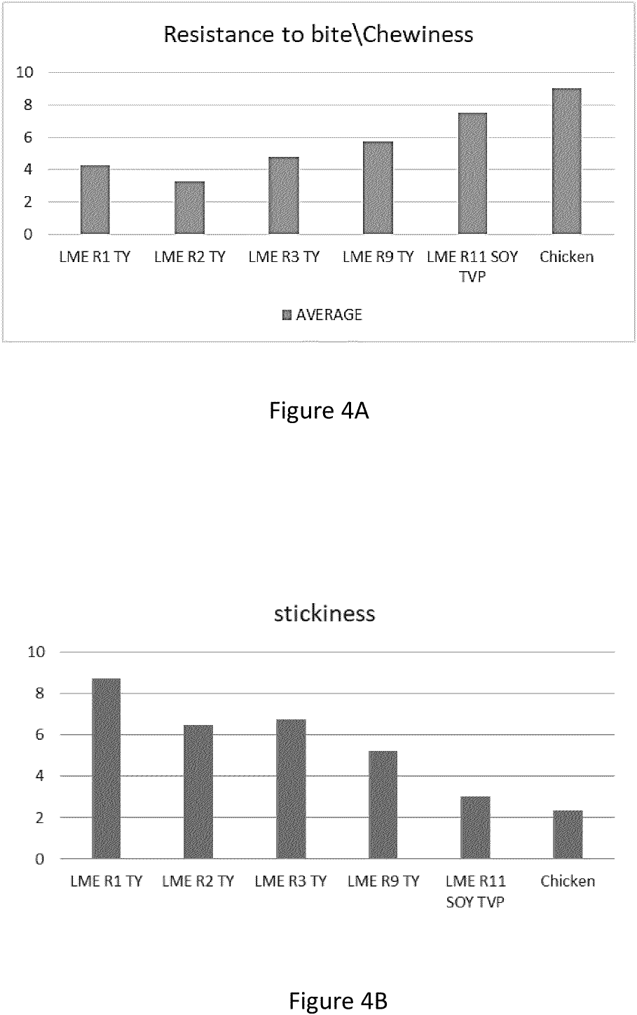

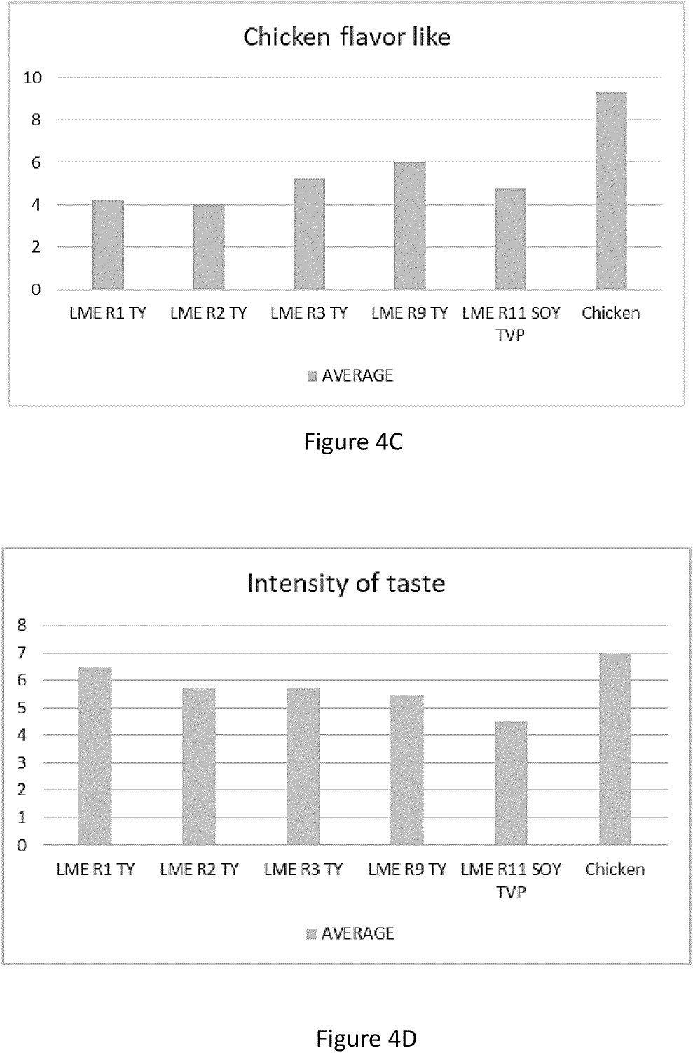

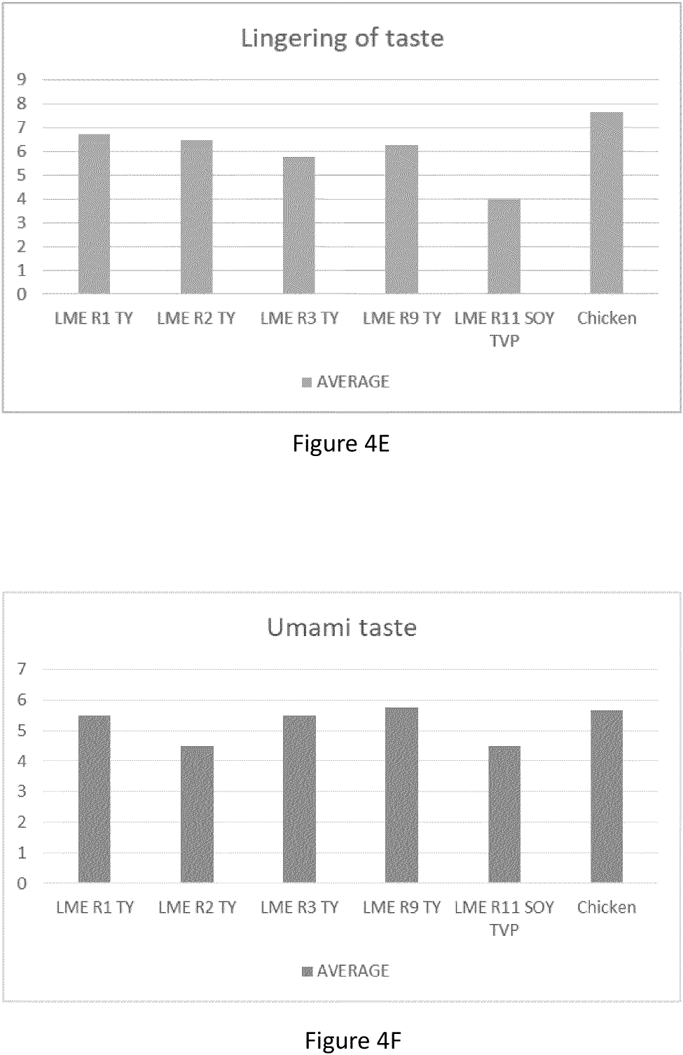

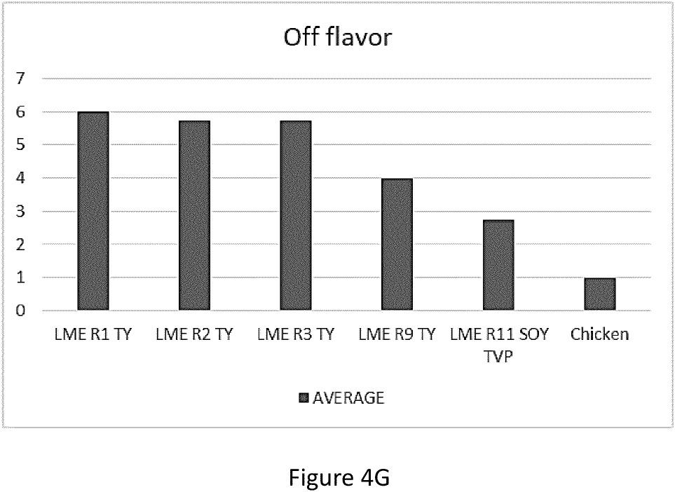

Sensory tests were conducted with a group of 4 people as tasters (FIGS. 4A-G). Results show that with all patty comprising yeast low moisture extrudates, the meaty flavors were closest to chicken: lingering of taste, intensity of taste and umami were rated similar to chicken.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An extrudate comprising a yeast material, wherein said yeast material constitutes between 35% and 100% by dry weight of said extrudate, wherein said yeast material comprises a protein content between 10% and 90% by weight of said yeast material.

2. (canceled)

3. The extrudate of claim 1 comprising at least 50% by weight of said yeast material.

4. The extrudate of claim 1, comprising between 50% and 99% by weight of said yeast material.

5. The extrudate of claim 1, comprising between 1% and 15% weight per weight (w/w) of water.

6. The extrudate of claim 1, further comprising between 10% and 40% (w/w) of a starch, a cereal, a grain, a legume, or any combination thereof.

7. The extrudate of claim 1, further comprising between 30% and 75% (w/w) of an additional protein, wherein said additional protein is a plant protein selected from the group consisting of: vegetable protein, a legume protein, a seed protein, a grain protein, a tuber protein, a root protein, a fruit protein, hemp protein, a nut protein, an algae protein, a seaweed protein, and any combination thereof.

8. The extrudate of claim 1, characterized by a fibrous texture.

9. The extrudate of claim 7,comprising between 30% and 75% (w/w) of water.

10. The extrudate of claim 1, comprising between 0.01% and 1.5% (w/w) of an expansion regulating agent.

11. The extrudate of claim 10, wherein said expansion regulating agent comprises calcium carbonate.

12. The extrudate of claim 1, further comprising a thickening agent, an emulsifier, coloring agent, flavoring agent, flavor masking agent, or any combination thereof.

13. The extrudate of claim 1, wherein said yeast material is derived from downstream food-related industries.

14. The extrudate of claim 1, wherein said yeast material is selected from the group consisting of: a whole yeast, a yeast biomass, a yeast filtrate, a yeast concentrate, any fraction thereof, and any combination thereof.

15. A food product comprising the extrudate of claim 1.

16. A food product consisting essentially of the extrudate of claim 1.

17. A food product characterized as being suitable for use as an equivalent product to meat, eggs, dairy products, or any combination thereof, comprising between 35% and 100% by dry weight of a yeast material, wherein said yeast material is selected from the group consisting of: a whole yeast, a yeast biomass, a yeast filtrate, a yeast concentrate, a yeast extrudate, any fraction thereof, and any combination thereof.

18. (canceled)

19. The food product of claim 15, wherein said food product is in the form of a nugget, a breast, a steak, or a minced meat-like product.

20. A method for improving the flavor of a yeast material, comprising contacting said yeast material with an agent, wherein said agent is characterized by being able to adjust the pH of said yeast material to a pH ranging from 4 to 7.5.

21. The method of claim 20, wherein said improving comprises reducing yeast taste, off-flavor of yeast taste, enhance meat flavor, or any combination thereof.
